(19) United States
(12) Patent Application Publication
(10) Pub. No.: US 2012/0034130 A1
(43) Pub. Date: Feb. 9, 2012

(54) STERILIZATION PROCESS WITH CONTROLLED COOLING

(75) Inventors: Bengt Storberg, Falkenberg (SE); Jan C. Nilsson, Heberg (SE)

(73) Assignee: GETINGE STERILIZATION AB, GETINGE (SE)

(21) Appl. No.: 13/138,858

(22) PCT Filed: May 8, 2009
(86) PCT No.: PCT/SE2009/050507

§ 371 Date: Oct. 11, 2011

(51) Int. Cl. A61L 2/04

(52) U.S. Cl. 422/1; 422/105

(57) ABSTRACT

The present invention relates to a process (100) for sterilization of prefilled containers in an autoclave chamber (1) and comprises a sterilization phase (102) and a cooling phase (103). The sterilization phase (102) comprises sterilizing prefilled containers at a temperature in the range of from 105 to 140° C. during a time sufficient to sterilize the content of said containers.

Furthermore, the present invention relates to an autoclave for use in a process for sterilization of prefilled containers.

### TECHNICAL FIELD

The present invention relates to a process for sterilization of prefilled containers in an autoclave chamber comprising a sterilization phase and a cooling phase. Furthermore, the invention relates to an autoclave for sterilization of prefilled containers.

### BACKGROUND OF THE INVENTION

Preparation of injectable medications and intravenous solutions requires a high sterility level to reduce or eliminate the risk of exposing persons and animals to microorganisms, such as bacteria and viruses. A widely used process for sterilization of prefilled containers is the autoclave, e.g. the steam/air autoclave and the circulating water autoclave.

Pre-filled plastic syringes, containers and vials comprising liquid, gel-like or paste-like pharmaceutical or biological products are typically placed in the autoclave and subjected to steam or super heated water until the temperature reaches about 121° C. This temperature is maintained for a time sufficient to sterilize the content.

As the prefilled container is heated, the fluid or formulation contained therein vaporizes to the saturated pressure, i.e. the maximum pressure at a given temperature where part of the fluid or formulation vaporizes. As the temperature increases, the internal pressure of the container; i.e. the sum of the initial pressure within the container and the saturated pressure, increases.

To avoid deformation caused by this internal pressure, a support pressure is typically introduced.

Following sterilization, the prefilled containers are subjected to a cooling step. During cooling, the temperature in the autoclave chamber drops such that the temperature within the container is substantially higher than the chamber temperature. As a consequence, a pressure differential between individual containers and the autoclave chamber is created.

While containers made of e.g. glass are able to withstand such pressure differentials, pre-filled syringes and containers made of plastic do not tolerate significant pressure differentials.

Before sterilization in an autoclave, several parameters must be extensively validated, such as the necessary sterilization time, the maximum temperature and pressure as well as the arrangement of the prefilled containers within the autoclave.

Typically, the warmest position in the chamber is identified, and a temperature sensor is placed in the container arranged in this position.

However, individual containers within the chamber may experience different temperatures, and during cooling, the pressure differential created between individual containers and the chamber may vary significantly. As a consequence, some containers are more susceptible to deformation. Furthermore, the temperature difference between individual containers may lead to variations in the F₀ value; i.e. the microbial inactivation capability, between individual containers. Products comprising active agents sensitive to overexposure of heat must be carefully monitored and it is important to keep the F₀ value of the individual containers consistent, i.e. within specific limits.

To date, the cooling phase of the sterilization process is often determined by choosing a suitable temperature gradient; i.e. a predetermined temperature decrease per time unit which may depend on the liquid being sterilized, fill volume and the container material.

However, the temperature and the pressure may still vary significantly between individual containers within the autoclave. As a consequence, the F₀ value may differ and some containers may still be susceptible to deformation during cooling.

### SUMMARY OF THE INVENTION

One object of the present invention is to provide a process for sterilization whereby deformation during cooling can be prevented while at the same time providing for a reduced variation in the Fₒ value among individual prefilled containers.

This object is achieved by a process for sterilization according to the appended claims.

Thus, in a first aspect the present invention relates to a process for sterilization of prefilled containers in an autoclave chamber comprising a sterilization phase and a cooling phase. The sterilization phase comprises sterilizing the prefilled containers at a temperature in the range of from 105 to 140° C. during a time sufficient to sterilize the content of the containers.

The cooling phase comprises the steps of:
monitoring a container temperature, T_{cont,max}, of the prefilled container expected to have the highest temperature of all containers present in the chamber
monitoring a chamber temperature, T_{ch}, at a given location within the chamber
controlling T_{ch} such that the temperature difference, ΔT=T_{cont}−T_{ch}, is kept below a predetermined value during the cooling phase.

Sterilization of prefilled containers in an autoclave typically comprises the steps of heating, sterilizing and cooling.

During heating and sterilization, a sterilization medium, such as steam/air mixtures or super heated water may be circulated throughout the goods to be sterilized. In the case of the sterilization medium being a steam/air mixture, the circulation may be e.g. accomplished by one or more fans mounted within the autoclave.

As the temperature rises, typically from room temperature to the sterilization temperature of from 105 to 140° C., usually 121° C., part of the content of the container vaporizes to the saturated pressure.

A support pressure is typically applied to avoid container explosion/deformation caused by the internal container pressure. The saturated vapor pressure is determined by the temperature within a container, and the desired level may be calculated by using e.g. steam tables.

After completion of the sterilization phase, which typically takes place during 10-20 min, the containers are subjected to a cooling phase. This may be achieved by adding cooling water to heating means, e.g. heat exchangers present in the autoclave in order to cool the sterilization medium.

In the process according to the invention, the temperature, T_{cont,max}, of the temperature of the prefilled container which is expected to have the highest temperature of all containers present in the chamber, is monitored.

During the validation process, which is typically performed for the autoclave before using the autoclave for a give sterilization process, the warmest position in the chamber is identified, and temperature sensing means is provided to sense the temperature of the container which is to be arranged in this position.

Simultaneously, a chamber temperature, T_{ch}, at a given location within the chamber may be monitored and the temperature difference, ΔT=T_{cont,max}−T_{ch}, can be calculated.

The present inventors have found that by controlling the temperature such that ΔT is kept below a predetermined value during the cooling phase, a more controlled cooling may be achieved.

When the temperature decreases during the cooling phase, the chamber temperature drops faster than the internal temperature of the prefilled containers. Accordingly, the chamber temperature will always be lower than the temperature within the containers.

By controlling the temperature of the sterilization medium in the chamber to maintain the temperature difference between the chamber and the container expected to have the highest temperature below a predetermined value, any temperature difference among the remaining containers will be kept within a specific range and below a predetermined value.

This has the advantage that the temperature differential between individual containers and the chamber will be kept more consistent.

As a consequence, the pressure differential created between individual containers and the chamber may be more accurately controlled, and container deformation can be prevented. Furthermore, the F₀ value; i.e. the microbial inactivation capability, between individual containers can be harmonized and can be kept within specific limits. This is highly advantageous, especially during sterilization of sensitive products being susceptible to overexposure of heat and which do not allow large variations in F₀ values.

The process of the invention allows for a safe and robust process which prevents deformation of containers during cooling.

In embodiments, ΔT may be less than 30° C., e.g. less than 10° C.

In embodiments, the step of controlling the chamber temperature, T_{ch}, comprises the steps of:

comparing T_{ch} with a first predetermined threshold temperature;

if T_{ch} is higher than the first threshold temperature, controlling T_{ch} such that the temperature difference is kept below a first predetermined value; and

if T_{ch} is lower than the first threshold temperature, controlling T_{ch} such that the temperature difference is kept below a second predetermined value, being higher than the first predetermined value.

After completion of the sterilization phase; i.e. upon initiation of the cooling phase the heated containers are more sensitive to variations in temperature and pressure than at the end of the cooling phase.

A first predetermined threshold temperature may be determined during the above-mentioned validation process. Depending on the products to be sterilized and the containers and container loads to be used, this first threshold temperature may vary.

In embodiments, the first predetermined threshold temperature may in the range of from 80 to 110° C.

When the chamber temperature is above the first threshold temperature; i.e. during a first part of the cooling phase, the temperature difference should be kept below a first predetermined value. During this first part, the temperature difference should be kept relatively small, since the containers are more susceptible to deformation and variations in F₀ values.

However, when the chamber temperature is below the first threshold temperature; i.e. during a second part of the cooling phase, the containers are less sensitive to temperature and pressure variations, and the temperature difference may thus be kept below a second predetermined value higher than the first predetermined value. Accordingly, the second part of the cooling phase may be accelerated. This allows for a more rapid cooling phase, and hence, a more rapid sterilization process.

In embodiments, the step of controlling T_{ch} may further comprise the steps of:

comparing T_{ch} with a second predetermined threshold temperature, being lower than the first predetermined threshold temperature,

if T_{ch} is lower than the second threshold temperature, controlling T_{ch} such that the temperature difference is kept below a third predetermined value, being higher than the second predetermined value.

A second predetermined threshold temperature may also be determined during the validation process.

Accordingly, below the second predetermined threshold temperature; i.e. during a third part of the cooling phase, the temperature difference between the container expected to have the highest temperature and the chamber may be substantially larger. The cooling phase may thus be accelerated further, and a more rapid sterilization process can thereby be achieved.

In another aspect, the present invention relates to an autoclave for use in the sterilization of prefilled containers. The autoclave comprises a chamber for receiving prefilled containers to be sterilized, as well as heating and cooling means for heating and cooling the prefilled containers.

Such heating and cooling means may e.g. be heat exchangers which supply either heating or cooling fluid depending on the phase of the sterilization process.

The autoclave further comprises a control unit configured to acquire temperature data from a first temperature sensing means sensing a container temperature, T_{cont,max}, of the one prefilled container expected to have the highest temperature of all prefilled containers in the chamber.

The control unit is further configured to acquire temperature data from a second temperature sensing means sensing a chamber temperature, T_{ch}, at a given location within the chamber.

The control unit is configured to control T_{ch} such that the temperature difference, ΔT=T_{cont}−T_{ch} is kept below a predetermined value during the cooling phase of the sterilization process.

An autoclave according to the present invention allows for a controlled cooling phase, wherein the F₀ value between individual containers is kept more consistent. Furthermore, deformation of pressure sensitive containers present in the chamber may be prevented and avoided.

In embodiments, the control unit may be adapted to control T_{ch} such that ΔT is less than 30°, e.g. less than 10° C.

This allows for an improved consistency of the internal pressure, and hence also the F₀ value, between individual containers in the chamber.

In embodiments, the control unit may be configured to control T_{ch} by comparing T_{ch} with a first predetermined threshold temperature;

if T_{ch} is higher than the first threshold temperature, controlling T_{ch} such that the temperature difference is kept below a first predetermined value; and

if T_{ch} is lower than the first threshold temperature, controlling T_{ch} such that the temperature difference is kept below a second predetermined value, being higher than the first predetermined value.

Accordingly, the cooling phase may be accelerated when the chamber temperature is lower than the first predetermined threshold temperature.

This embodiment allows for a more rapid sterilization process.

The temperature difference in this second part may thus be larger than the temperature difference of the first part, the latter being more susceptible to large temperature differences, and thus pressure differentials.

In embodiments, the first predetermined threshold temperature may be in the range of from 80 to 110° C.

In order to further speed up the sterilization process, the control unit may be adapted to further control T_{ch} by:

comparing T_{ch} with a second predetermined threshold temperature, being lower than the first predetermined threshold temperature,

if T_{ch} is lower than the second threshold temperature, controlling T_{ch} such that the temperature difference is kept below a third predetermined value, being higher than the second predetermined value.

Accordingly, when the chamber temperature is below the second predetermined threshold temperature; i.e. during a third part of the cooling phase, the cooling may be accelerated since the temperature difference may be much larger than in the first two parts of the cooling phase.

According to a further aspect, the above-mentioned and other objects are also achieved by a computer program configured to execute the steps of the process according to the invention when run on the control unit comprised in the autoclave according to the invention.

These and other aspects of the invention will be apparent from and elucidated with reference to the embodiment(s) described hereinafter.

### BRIEF DESCRIPTION OF THE DRAWINGS

FIG. 1 illustrates a steam/air autoclave according to an embodiment of the present invention; and

FIG. 2 is a diagram showing a sterilization process according to an embodiment of the present invention, that may be carried out in the autoclave in FIG. 1.

### DETAILED DESCRIPTION OF THE AN EMBODIMENT OF THE INVENTION

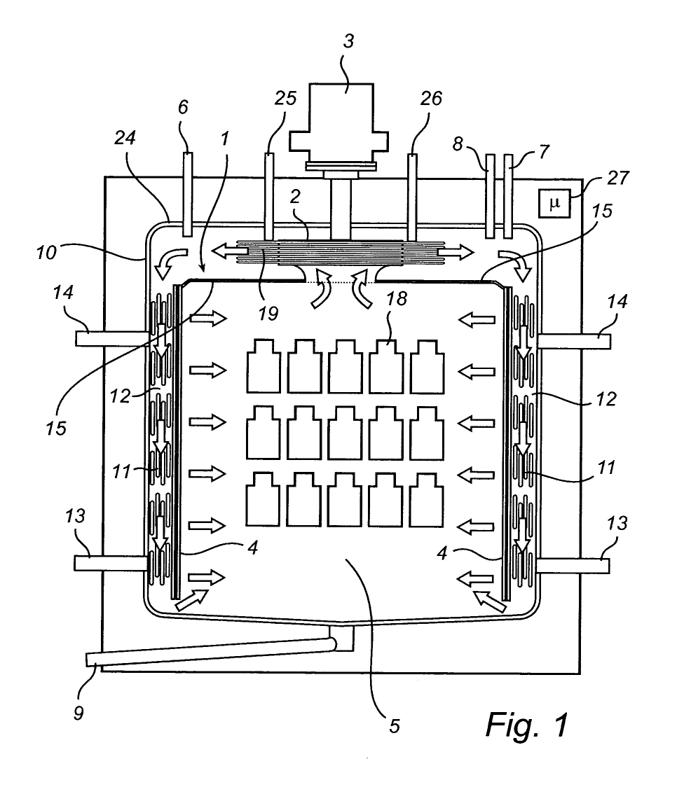

Sterilization using the process according to the present invention may e.g. be accomplished in a steam/air autoclave (or ventilator autoclave) as shown in FIG. 1.

The steam/air autoclave comprises a sterilization chamber 1 and a fan 2 for circulating the steam/air in the chamber 1. The autoclave is provided with a pipe 7 that supplies steam to the chamber 1, and a pipe 8 which supplies air to the chamber 1. A drainage 9 is arranged at the bottom of the chamber 1. The autoclave also comprises a safety valve 6 provided at the top of the autoclave

Partition walls or liners 4 are installed within the chamber 1, and these form a space 5 for goods 18 to be sterilized. The partition walls are arranged such that a duct in the form of a space 12 is created between each of the outer side walls 10 of the chamber 1 and the partition walls 4. The partition walls 4 leave an opening close to the floor of the autoclave. First heat exchangers 11 are provided in each of the ducts 12. The first heat exchangers are connected to pipes 13, 14 for supply and discharge of fluid, which may be either cooling or heating fluid.

This design ensures that the air/steam is routed in a circulation pattern from the top, down the sides (over the heat exchangers and in contact with the chamber walls) and up through the load, back to the fan inlet. This circulation pattern assures homogeneity and uniformity of temperature distribution, together with an optimized, rapid process.

The fan 2 may be driven by an electric motor 3 and may be mounted in the chamber ceiling 24 behind a partition wall top section 15 that is provided at a distance from the top wall of the autoclave. The partition wall top section 15 comprises at least one grid or net, which allows fluid in the space 5 to be drawn from the space 5 through the net by the fan 2.

The fan 2 directs steam/air towards the first heat exchangers 11, which serve to heat or cool the steam/air depending on the phase of the sterilization process.

In embodiments, the autoclave may comprise a second heat exchanger 19 arranged adjacent and circumscribing the fan 2. The second heat exchanger 19 may comprise a plurality of spiral-wounded interconnected horizontally provided pipes. Heating or cooling liquid may be circulated through the pipes from an external source through the pipes 25, 26 in order to cool or heat the steam/air being circulated in the chamber.

The autoclave has been described in relation to an exemplified embodiment, but several modifications and adaptations are possible.

It should be noted that the autoclave described above is suitable for use with the sterilization process according to the present invention, but the invention is not limited thereto. Any steam/air autoclave, ventilator autoclave or circulating water autoclave may be used.

When the goods to be sterilized are biological or pharmaceutical products, such as liquid, gel-like or paste-like pharmaceutical agents, injection fluids, culture media etc, these are typically prefilled into containers, and sterilized within the final containers.

The containers may e.g. be syringes, vials, cartridges, blister packs and are typically made of plastic or polymeric materials such as polyethylene or polypropylene, or co-polymers thereof.

Before the sterilization starts, parameters such as the necessary sterilization time, the maximum temperature and pressure as well as the arrangement of the prefilled containers within the autoclave are carefully validated. The warmest position in the chamber is identified, and a temperature sensor is placed in the container arranged in this position.

Individual containers within the chamber may, however, experience different internal temperatures, and this may result in a that the pressure differential; i.e. the pressure difference between the chamber and individual containers varies significantly.

As a consequence, some containers are more susceptible to deformation, and the F₀ value may vary significantly from one container to another.

The process according to the present invention allows for the prevention of deformation during the cooling phase and provides for a consistency in F₀ values between individual containers.

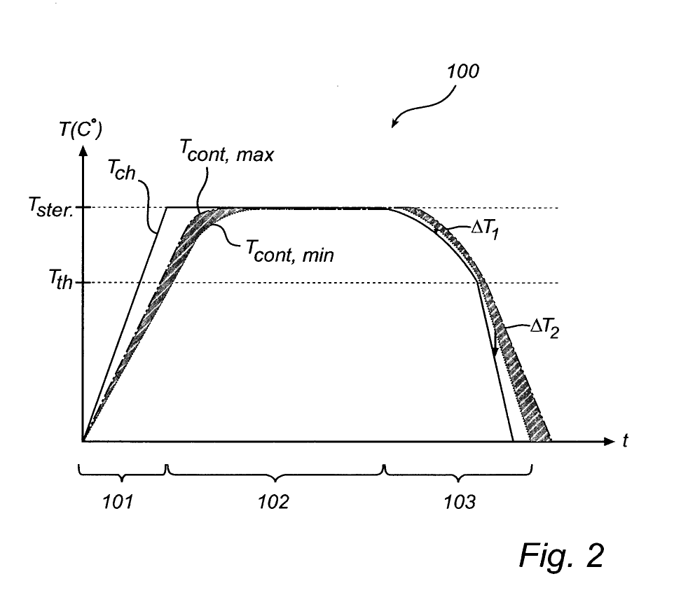

A sterilization process according to an exemplary embodiment of the present invention will now be described with reference to FIG. 2.

FIG. 2 is a diagram schematically illustrating an exemplary sterilization process 100 carried out in the ventilator autoclave shown in FIG. 1. The sterilization process 100 in FIG. 2 comprises a heating phase 101, a sterilization phase 102, and a cooling phase 103 as is schematically indicated in FIG. 2.

During the heating phase 101, the temperature of the sterilization medium in the chamber 1 is increased from the ambient temperature to the desired sterilization temperature Tₛₜₑᵣ. In the ventilator autoclave in FIG. 1, the heating may typically be carried out by releasing steam into the chamber 1, and the sterilization medium in the ventilator autoclave is thus a steam/air mixture. When the temperature T_{ch} of the steam/air mixture within the chamber 1 has reached the desired sterilization temperature Tₛₜₑᵣ, possibly with some ‘over-shoot’ to more quickly bring the containers 18 in the chamber 1 to the desired sterilization temperature Tₛₜₑᵣ, the temperature in the chamber 1 is kept constant during the sterilization phase 102.

As is schematically indicated by the curves labeled T_{cont,max} and T_{cont,min}, representing the temperatures of the containers expected to have the highest temperature and the lowest temperature, respectively, there will be a delay in the heating of the containers 18, and, at a given time during the heating phase 101, there will be a temperature spread among the containers This temperature spread is indicated by the hatched area between the curves labeled T_{cont,max} and T_{cont,min} in FIG. 2.

During the sterilization phase 102 the prefilled containers 18 are kept at a sterilization temperature Tₛₜₑᵣ in the range of from 105 to 140° C. during a time sufficient to sterilize the content of the containers.

Normally, the sterilization phase is carried out at 121° C. for e.g. 10-20 minutes. Potential pathogens present within the containers are thus killed.

A support pressure is typically used to avoid deformation/explosion caused by the internal container pressure.

The next phase of the sterilization process 100 is the cooling phase 103. In the cooling phase 103, the temperature T_{cont,max} of the prefilled container 18 expected to have the highest temperature is measured, and the temperature T_{ch} of the sterilization medium in the chamber 1 is controlled to maintain a first temperature difference ΔT₁. The first temperature difference ΔT₁ is selected to be sufficiently small so that deformation of the ‘warmest’ container 18 can be avoided during the cooling phase. The first temperature difference ΔT₁ may be selected depending on the container material and various other parameters, such as the wall thickness of the containers 18, the material properties of the container material, notably the softening temperature of the material, etc. In addition to allowing for avoidance of container deformation, the process according to the various embodiments of the present invention provides for maintaining a relatively small temperature spread (viz. below ΔT₁) between the warmest container and the coldest container in the chamber. Hereby, the important parameter F₀ can be kept controlled and the quality of the sterilization improved and ensured.

During the cooling, which is typically achieved by supplying cold water to the heat-exchangers 11, whereby the steam/air mixture in the chamber 1 is cooled, the temperature T_{ch} of the sterilization medium in the chamber 1 may be compared with a threshold temperature Tₜₕ. The threshold temperature may advantageously be chosen depending on the material properties of the container material, so that a larger pressure differential between the inside and the outside of the containers 18 is allowable below the threshold temperature Tₜₕ.

When the temperature T_{ch} of the sterilization medium in the chamber 1 drops below the threshold temperature Tₜₕ, the temperature T_{ch} of the sterilization medium (which is also sometimes referred to herein as the temperature within the chamber 1), the temperature T_{ch} of the sterilization medium is controlled to maintain a second temperature difference ΔT₂ between the temperature T_{ch} within the chamber 1 and the temperature T_{cont,max} of the ‘warmest’ container 18. This reduces the total time of the sterilization process 100 without compromising the sterilization quality, or risking deformation of the containers 18.

As was described above, the temperature, T_{cont,max}, of the prefilled container expected to have the highest temperature of all containers present in the chamber is monitored. Since the warmest position in the chamber is identified during the validation process, temperature sensing means is provided in the container localized in this position.

Simultaneously, the temperature within the chamber, T_{ch}, may be monitored and this is typically achieved by temperature sensing means provided at a given location in the autoclave chamber.

Referring again to FIG. 1, the autoclave further comprises a control unit 27 configured to acquire temperature data from a first temperature sensor (not shown) which senses a container temperature, T_{cont,max}, of the prefilled container expected to have the highest temperature of all prefilled containers in the chamber 1.

The control unit 27 is further configured to acquire temperature data from a second temperature sensor (not shown) which senses the chamber temperature, T_{ch}.

During the sterilization process 100 described above in connection with FIG. 2, the control unit 27 controls the chamber temperature by controlling various parts of the ventilator autoclave, such as the fan motor 3, the supply of cooling water to the heat exchangers 1, the supply of steam and/or air through the respective inlets 7, 8.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. For example, the present invention is not limited to the use of a specific autoclave, but may be used in e.g. a steam/air autoclave or a circulating water autoclave. Neither is the invention limited to a specific type of container or formulation, but pressure sensitive containers and sensitive biological or pharmaceutical formulations are advantageously sterilized by the process of the invention.

1. A process for sterilization of prefilled containers in an autoclave chamber, the process comprising a sterilization phase and a cooling phase, wherein said sterilization phase comprises sterilizing said prefilled containers at a sterilization temperature Tₛₜₑᵣ in the range of from 105 to 140° C. during a time sufficient to sterilize the content of said containers, and wherein said cooling phase comprises the steps of:
monitoring a container temperature, T_{cont,max}, of the one of said prefilled containers expected to have the highest temperature of all containers present in said chamber
monitoring a chamber temperature, T_{ch}, at a given location within said chamber
controlling the chamber temperature, T_{ch}, such that the temperature difference, ΔT=T_{cont}−T_{ch} is kept below a predetermined value during said cooling phase.

2. The process according to claim 1, wherein ΔT is less than 30° C.

3. The process according to claim 2, wherein ΔT is less than 10° C.

4. The process according to claim 1, wherein said step of controlling the chamber temperature, T_{ch}, comprises the steps of:
comparing Tch with a first predetermined threshold temperature;
if T_{ch} is higher than said first threshold temperature, controlling T_{ch} such that the temperature difference ΔT is kept below a first predetermined value; and
if T_{ch} is lower than said first threshold temperature, controlling T_{ch} such that the temperature difference ΔT is kept below a second predetermined value, being higher than said first predetermined value.

5. The process according to claim 4, wherein said first predetermined threshold temperature is in the range of from 80 to 110° C.

6. The process according to claim 4, wherein said step of controlling the chamber temperature, T_{ch}, further comprises the steps of:
comparing T_{ch} with a second predetermined threshold temperature, being lower than said first predetermined threshold temperature; and
if T_{ch} is lower than said second threshold temperature, controlling T_{ch} such that the temperature difference ΔT is kept below a third predetermined value, being higher than said second predetermined value.

7. An autoclave for use in a process for sterilization of prefilled containers, the autoclave comprising a chamber for receiving prefilled containers to be sterilized, heating and cooling means for heating and cooling said prefilled containers, wherein said autoclave further comprises a control unit configured to acquire temperature data from a first temperature sensor for sensing a container temperature, T_{cont,max}, of the one of said prefilled containers expected to have the highest temperature of all prefilled containers in the chamber, and a second temperature sensor for sensing a chamber temperature, T_{ch}, at a given location within the chamber, wherein said control unit is configured to control T_{ch} such that the temperature difference, ΔT=_{cont,max}−T_{ch} is kept below a predetermined value during the cooling phase of the sterilization process.

8. The autoclave according to claim 7, wherein said control unit is configured to control T_{ch} such that ΔT is less than 30° C.

9. The autoclave according to claim 8, wherein said control unit is configured to control T_{ch} such that ΔT is less than 10° C.

10. The autoclave according to claim 7, wherein said control unit is configured to:
compare T_{ch} with a first predetermined threshold temperature;
if T_{ch} is higher than said first threshold temperature, control T_{ch} such that the temperature difference ΔT is kept below a first predetermined value; and
if T_{ch} is lower than said first threshold temperature, control T_{ch} such that the temperature difference ΔT is kept below a second predetermined value, being higher than said first predetermined value.

11. The autoclave according to claim 10, wherein said first predetermined threshold temperature is in the range of from 80 to 110° C.

12. The autoclave according to claim 10, wherein said control unit is further configured to:
compare Tch with a second predetermined threshold temperature, being lower than said first predetermined threshold temperature,
if T_{ch} is lower than said second threshold temperature, control T_{ch} such that the temperature difference is kept below a third predetermined value, being higher than said second predetermined value.

13. A computer program configured to execute the steps of the process according to claim 1.
